(19) United States
(12) Patent Application Publication
(10) Pub. No.: US 2025/0194483 A1
(43) Pub. Date: Jun. 19, 2025

(54) BIOLOGICAL METHOD FOR DECARBOXYLATION OF CANNABINOIDS DIRECTLY IN PLANT TISSUES

(71) Applicant: ALKION BIOINNOVATIONS, Boulogne-Billancourt (FR)

(72) Inventors: Houssein WEHBI, Strasbourg (FR); Sarah-Meryll BUET, Boulogne-Billancourt (FR); Julien SECHET, Saint Cyr L'ecole (FR)

(73) Assignee: ALKION BIOINNOVATIONS, Boulogne-Billancourt (FR)

(21) Appl. No.: 18/847,912

(22) PCT Filed: Mar. 17, 2023
(86) PCT No.: PCT/EP2023/056850

§ 371 Date: Sep. 17, 2024

(30) Foreign Application Priority Data
Mar. 18, 2022 (EP) 22305320.8

(51) Int. Cl. A01H 4/00; A01H 5/12; A01H 6/28; C12N 1/16; C12N 1/20; C12P 7/22; C12P 17/06

(52) U.S. Cl. CPC A01H 4/00; A01H 5/12; A01H 6/28; C12N 1/16; C12N 1/20; C12P 7/22; C12P 17/06

(57) ABSTRACT

In vitro production of acidic cannabinoids and their bioconversion to non-acidic form by microorganisms via a method for producing plant tissues containing one or more decarboxylated cannabinoid(s) by (a) inoculating a temporary liquid immersion culture system containing a sterile liquid culture medium containing at least one cytokinin and/or at least one auxin with sterile plant material from a Cannabis sativa cultivar; (b) cultivating the plant material in the temporary liquid immersion culture system under conditions suitable for growing plant biomass; (c) inoculating the sterile liquid culture medium with one or more bacterial and/or yeast strain(s), (d) cultivating the plant material in the temporary liquid immersion culture system inoculated by the bacterial or yeast strain(s), and (e) collecting plant tissues. Also via using one or more bacterial and/or yeast strain(s) for decarboxylating one or more cannabinoid(s) in plant tissues of Cannabis sativa cultivars cultivated in a temporary liquid immersion culture system.

### TECHNICAL FIELD OF THE INVENTION

The present invention is in the field of in vitro production of acidic cannabinoids and their bioconversion to non-acidic form by microorganisms. It relates to a method for producing plant tissues containing one or more decarboxylated cannabinoid(s), the method comprising: a) inoculating a temporary liquid immersion culture system containing a sterile liquid culture medium containing at least one cytokinin and/or at least one auxin with sterile plant material from a Cannabis sativa cultivar; b) cultivating the plant material in the temporary liquid immersion culture system under conditions suitable for growing plant biomass; c) inoculating the sterile liquid culture medium with one or more bacterial and/or yeast strain(s), d) cultivating the plant material in the temporary liquid immersion culture system inoculated by the bacterial or yeast strain(s), optionally under abiotic stress, and e) collecting plant tissues containing one or more decarboxylated cannabinoid(s). It further relates to a method for producing one or more decarboxylated cannabinoid(s) based on the previous method, and to the use of one or more bacterial and/or yeast strain(s) for decarboxylating one or more cannabinoid(s) in plant tissues of Cannabis sativa cultivars cultivated in a temporary liquid immersion culture system, wherein the one or more bacterial and/or yeast strain(s) are co-cultivated with the Cannabis sativa cultivars in the temporary liquid immersion culture system.

### BACKGROUND ART

Various compounds of interest were originally isolated from plants or microorganisms and are still obtained from them. However, for some compounds, the extracted product requires a transformation to reach its active form. Cannabinoids are a good example and their different forms and isomers have received a lot of attention lately. Naturally, the plant produces acidic cannabinoids such as tetrahydrocannabinolic acid (THCA), cannabidiolic acid (CBDA), cannabichromenolic acid (CBCA) or cannabigerolic acid (CBGA), but the active pharmaceutical compounds tested, evaluated and approved as drugs are the decarboxylated forms (neutral forms) such as tetrahydrocannabinol (THC), cannabidiol (CBD), cannabichromene (CBC) or cannabigerol (CBG) (Lewis-Bakker et al. Cannabis and cannabinoid research, 4(3), 183-194; Pertwee, R. (2008). British journal of pharmacology, 153(2), 199-215; McPartland, J. M. et al (2017). Cannabis and cannabinoid research, 2(1), 87-95).

The process commonly used to obtain cannabinoids in their decarboxylated form is heating the cannabis extract (Wang, M. et al (2016). Cannabis and cannabinoid research, 1(1), 262-271; Veress, T. et al. (1990). Journal of Chromatography A, 520, 339-347). For recreational use of cannabis flowers, the decarboxylation process relies on the combustion of a cigarette or the use of other smoking methods. Relatively speaking, low-temperature heating results in long reaction times. The decarboxylation of THCA into the psychoactive form, THC, would require 3 hours at 100° C. and 4 hours at 98° C. At high temperatures, between 160° C. and 200° C., only 10 minutes to a few seconds respectively are needed to completely convert THCA into THC (Iffland, K., Carus, M., &t Grotenhermen, F. (2016). Decarboxylation of Tetrahydrocannabinolic acid (THCA) to active THC. European Industrial Hemp Association). Although high-temperature heating reduces the time and transform up to 99% of acid forms (Wang, M. et al (2016). Cannabis and cannabinoid research, 1(1), 262-271), it results in undesirable cannabinoid degradation. Indeed, at a temperature allowing the full transformation of CBGA, only 47.3% is recovered as CBG, the rest being degraded (Wang, M. et al (2016). Cannabis and cannabinoid research, 1(1), 262-271). Degradation metabolites such as CBN are further produced during the heating process (Hazekamp, A., et al (2004). Journal of liquid chromatography & related technologies, 27(15), 2421-2439), which is thus not optimal. A controlled process for the decarboxylation of cannabis has been described, in which THCA and other acidic forms of cannabinoids are decarboxylated through a chemical reaction. This process requires several heating steps and the addition of different elements such as solvent and cofactor to be achieved (US20120046352). Some of these elements can be toxic and expensive. In addition, while the rate of cannabinoid degradation is not mentioned in this document, it is known in the art that heating induces degradation of cannabinoids. Another decarboxylation method uses microwave treatment under atmospheric pressure or vacuum to provide rapid decarboxylation with high yields (Lewis-Bakker, M. M., et al. (2019). Cannabis and cannabinoid research, 4(3), 183-194). Decarboxylation yields obtained with this method reach up to 92% for THC (US20190038663) and 92.8% for CBD (US20210100864). It however requires expensive microwave equipment and induces degradation of other metabolites. This method is thus also not optimal.

Furthermore, decarboxylation of THCA and CBDA have been described with these methods, however CBCA decarboxylation is not described in the literature, neither using high temperature heating nor microwave treatment. Therefore, the reaction conditions are not optimal, and the known processes use high pressures and/or solvents to achieve a more optimal yield, but this often results in increased processing time. Moreover, the known methods generally use potentially toxic and/or expensive materials and induce some degradation of cannabinoids, thus altering the final yield in decarboxylated cannabinoids.

Therefore, there is a need for new methods of producing pharmaceutically active decarboxylated (neutral) cannabinoids that would be fast, not consume a lot of energy, would not need the use of cofactors, would be solvent-free and, most importantly, would not degrade the cannabinoids.

### SUMMARY OF THE INVENTION

In the context of the present invention, the inventors surprisingly found that when Cannabis sativa cultivars are cultivated in a temporary immersion bioreactor (TIB) in the presence of one or more bacterial and/or yeast strain(s), plant tissue collected from the cultivated Cannabis sativa cultivars naturally contain a high amount and proportion of decarboxylated (neutral) cannabinoids. Therefore, by simply adding one or more bacterial and/or yeast strain(s) to Cannabis sativa cultivars cultivated in a TIB, cannabinoids may be naturally and biologically decarboxylated, without cannabinoid degradations and without the necessity of additional energy, cofactors or solvents. This is a completely different approach compared to the physical and/or chemical methods known in the art.

In a first aspect, the present invention thus relates to a method for producing plant tissues containing one or more decarboxylated cannabinoid(s), the method comprising:
a) inoculating a temporary liquid immersion culture system containing a sterile liquid culture medium containing at least one cytokinin and/or at least one auxin with sterile plant material from a Cannabis sativa cultivar;
b) cultivating the plant material in the temporary liquid immersion culture system under conditions suitable for growing plant biomass;
c) inoculating the sterile liquid culture medium with one or more bacterial and/or yeast strain(s);
d) cultivating the plant material in the temporary liquid immersion culture system inoculated by the one or more bacterial and/or yeast strain(s), optionally under abiotic stress; and
e) collecting plant tissues containing one or more decarboxylated cannabinoid(s).

The present invention also relates to a method for producing one or more decarboxylated cannabinoid(s), the method comprising:
a) producing plant tissues containing one or more decarboxylated cannabinoid(s) using the method according to the invention,
b) collecting or extracting the one or more decarboxylated cannabinoid(s) from the obtained plant tissues, and optionally from the culture medium of the temporary liquid immersion culture system,
c) optionally, purifying the collected or extracted one or more decarboxylated cannabinoid(s), and
d) optionally, adding an acceptable diluent, excipient or carrier to the one or more decarboxylated cannabinoid(s).

The present invention also relates to the use of one or more bacterial and/or yeast strain(s) for decarboxylating one or more cannabinoid(s) in plant tissues of Cannabis sativa cultivars cultivated in a temporary liquid immersion culture system, wherein the one or more bacterial and/or yeast strain(s) are co-cultivated with the Cannabis sativa cultivars in the temporary liquid immersion culture system.

### DESCRIPTION OF THE FIGURES

FIG. 1. Percentage of decarboxylated cannabinoids (THC and CBC) detected in plant tissues of Cannabis sativa cultivar Jamaican Pearl (JP) cultivated in RITA® TIB under the following conditions: Contaminated (4 weeks culture in RITA® TIB contaminated by Curtobacterium spp. (Bacteria)); Control (4 weeks culture in non-contaminated RITA® TIB) or Contaminated+abiotic stress (4 weeks culture in RITA® TIB contaminated by Curtobacterium spp. (Bacteria)+3 further weeks in RITA® TIB contaminated by Curtobacterium spp. (Bacteria) under abiotic stress due to cessation of immersion). See more precise culture parameters (medium, immersion, light conditions and temperature in Example 1).

FIG. 2. Percentage of decarboxylated cannabinoids (THC and CBC) detected in plant tissues of Cannabis sativa cultivar California indica (CL) cultivated in RITA® TIB under the following conditions: Contaminated (4 weeks culture in RITA® TIB contaminated by Staphylococcus epidermidis); Control (4 weeks culture in non-contaminated RITA® TIB) or Contaminated+abiotic stress (4 weeks culture in RITA® TIB contaminated by Staphylococcus epidermidis+3 further weeks in RITA® TIB contaminated by Staphylococcus epidermidis under abiotic stress due to cessation of immersion). See more precise culture parameters (medium, immersion, light conditions and temperature in Example 1).

FIG. 3. Percentage of decarboxylated cannabinoids (THC, CBC and CBD) detected in plant tissues of Cannabis sativa cultivar Cannatonic (CN-B) cultivated in RITA® TIB under the following conditions: Contaminated (4 weeks culture in RITA® TIB contaminated by Micrococcus luteus (Bacteria)); Control (4 weeks culture in non-contaminated RITA® TIB) or Contaminated+abiotic stress (4 weeks culture in RITA® TIB contaminated by Micrococcus luteus (Bacteria)+3 further weeks in RITA® TIB contaminated by Micrococcus luteus under abiotic stress due to cessation of immersion). See more precise culture parameters (medium, immersion, light conditions and temperature in Example 1).

FIG. 4. Percentage of decarboxylated cannabinoids (THC, CBC and CBD) detected in plant tissues of Cannabis sativa cultivar Cannatonic (CN-B) cultivated in RITA® TIB under the following conditions: Contaminated (4 weeks culture in RITA® TIB contaminated by Microbacterium ginsengisoli (Bacteria)); Control (4 weeks culture in non-contaminated RITA® TIB) or Contaminated+abiotic stress (4 weeks culture in RITA® TIB contaminated by Microbacterium ginsengisoli (Bacteria)+3 further weeks in RITA® TIB contaminated by Microbacterium ginsengisoli (Bacteria) under abiotic stress due to cessation of immersion). See more precise culture parameters (medium, immersion, light conditions and temperature in Example 1).

FIG. 5. Percentage of decarboxylated cannabinoids (THC and CBC) detected in plant tissues of Cannabis sativa cultivar Amazing Haze (AH) cultivated in RITA® TIB under the following conditions: Contaminated (4 weeks culture in RITA® TIB contaminated by Cystobasidium minutum (Yeast)); Control (4 weeks culture in non-contaminated RITA® TIB) or Contaminated+abiotic stress (4 weeks culture in RITA® TIB contaminated by Cystobasidium minutum (Yeast)+3 further weeks in RITA® TIB contaminated by Cystobasidium minutum (Yeast) under abiotic stress due to cessation of immersion). See more precise culture parameters (medium, immersion, light conditions and temperature in Example 1).

FIG. 6. Percentage of decarboxylated cannabinoids (THC and CBC) detected in plant tissues of Cannabis sativa cultivar Cannatonic (CN-A) cultivated in RITA® TIB under the following conditions: Control (4 weeks culture in non-contaminated RITA® TIB); Abiotic stress (4 weeks culture in RITA® TIB+3 further weeks in RITA® under abiotic stress due to cessation of immersion); Contaminated+abiotic stress (4 weeks culture in RITA® TIB contaminated by Roseomonas mucosa (Bacteria)+3 further weeks in RITA® TIB under abiotic stress due to cessation of immersion). See more precise culture parameters (medium, immersion, light conditions and temperature in Example 2).

FIG. 7. Percentage of decarboxylated cannabinoids (THC and CBC) detected in plant tissues of Cannabis sativa cultivar Cannatonic (CN-A) cultivated in RITA® TIB for 7 weeks (49 days) under the following conditions: Co-culture for 44 days (5 days plant culture in RITA® TIB contaminated by Roseomonas mucosa (Bacteria)); Co-culture for 23 days+21 days abiotic stress (5 days plant culture in RITA® TIB contaminated by Roseomonas mucosa (Bacteria) for 23 days+3 weeks in RITA® TIB under abiotic stress due to cessation of immersion); Co-culture for 30 days (19 days plant culture in RITA® TIB contaminated by Roseomonas mucosa (Bacteria)) or Co-culture for 9 days+21 days abiotic stress (19 days plant culture in RITA® TIB contaminated by Roseomonas mucosa (Bacteria) for 9 days+3 weeks in RITA® TIB under abiotic stress due to cessation of immersion). The optical density (OD) of the bacteria used in this experiment is between 0.135 and 0.238. The final OD of the bacteria in the medium is 0.00574; See more precise culture parameters (medium, immersion, light conditions and temperature in Example 2).

FIG. 8. Total cannabinoids content in plant tissues of Cannabis sativa cultivar Cannatonic (CN-A) cultivated in RITA® TIB for 7 weeks (49 days) under the following conditions: Co-culture for 44 days (5 days plant culture in RITA® TIB contaminated by Roseomonas mucosa (Bacteria)); Co-culture for 23 days+21 days abiotic stress (5 days plant culture in RITA® TIB contaminated by Roseomonas mucosa (Bacteria) for 23 days+3 weeks in RITA® TIB under abiotic stress due to cessation of immersion); Co-culture for 30 days (19 days plant culture in RITA® TIB contaminated by Roseomonas mucosa (Bacteria)) or Co-culture for 9 days+21 days abiotic stress (19 days plant culture in RITA® TIB contaminated by Roseomonas mucosa (Bacteria) for 9 days+3 weeks in RITA® TIB under abiotic stress due to cessation of immersion). The optical density (OD) of the bacteria used in this experiment is between 0.135 and 0.238. The final OD of the bacteria in the medium is 0.00574; See more precise culture parameters (medium, immersion, light conditions and temperature in Example 2).

### DETAILED DESCRIPTION OF THE INVENTION

### Method for Producing Plant Tissues Containing One or More Decarboxylated Cannabinoid(s)

The present invention first relates to a method for producing plant tissues containing one or more decarboxylated cannabinoid(s), the method comprising:
a) inoculating a temporary liquid immersion culture system containing a sterile liquid culture medium containing at least one cytokinin and/or at least one auxin with sterile plant material from a Cannabis sativa cultivar;
b) cultivating the plant material in the temporary liquid immersion culture system under conditions suitable for growing plant biomass;
c) inoculating the sterile liquid culture medium with one or more bacterial and/or yeast strain(s);
d) cultivating the plant material in the temporary liquid immersion culture system inoculated by the one or more bacterial and/or yeast strain(s), optionally under abiotic stress; and
e) collecting plant tissues containing one or more decarboxylated cannabinoid(s).

### Decarboxylated Cannabinoids

More than 130 distinct cannabinoid compounds have been described as present in plants of the genus Cannabis (Berman et al., 2018. Sci Rep. 8(1), 14280). Most cannabinoids exist in two forms:
“acidic cannabinoids”, including tetrahydrocannabinolic acid (THCA), cannabidiolic acid (CBDA), cannabichromenolic acid (CBCA) and cannabigerolic acid (CBGA); and
“decarboxylated cannabinoids” (also referred to as “neutral cannabinoids”), including tetrahydrocannabinol (THC), cannabidiol (CBD), cannabichromene (CBC) or cannabigerol (CBG).

The acid form is the one usually found in nature, designated by an “A” at the end of its acronym (i.e. THCA is the acidic form of THC), although the pharmaceutically active form is generally the decarboxylated form.

The method for producing plant tissues containing one or more decarboxylated cannabinoid(s) according to the invention has been found to permit natural decarboxylation of several acidic cannabinoids, including THCA, CBCA and CBDA, into their decarboxylated (or neutral) form (THC, CBC and CBD, respectively). Its ability to naturally decarboxylate other acidic cannabinoids has not yet been tested, but based on the effect obtained on THCA, CBCA and CBDA and on the common mechanism (decarboxylation), it may be expected to work also for other acidic cannabinoids.

However, in a preferred embodiment, the method is for producing plant tissues containing THC, CBC, CBD, or any combination thereof. In particular, the method may be for producing plant tissues containing: THC; CBC; CBD; THC and CBC; or THC, CBC and CBD.

Step a)—Plant Inoculation in a Temporary Liquid Immersion Culture System

### Sterile Plant Material

A sterile plant material from a Cannabis sativa cultivar is inoculated in a temporary liquid immersion culture system containing a sterile liquid culture medium containing at least one cytokinin.

As demonstrated in experimental examples below, the method for producing plant tissues containing one or more decarboxylated cannabinoid(s) according to the invention works for several distinct Cannabis sativa cultivars. In addition, as the same mechanism of action is expected to be needed for decarboxylation of acidic cannabinoids for any Cannabis sativa cultivar, the method for producing plant tissues containing one or more decarboxylated cannabinoid(s) according to the invention may be expected to work for any Cannabis sativa cultivar.

However, based on the experimental examples below, the method for producing plant tissues containing one or more decarboxylated cannabinoid(s) according to the invention may preferably be performed using the following Cannabis sativa cultivars: Jamaican Pearl (also referred to as “JP”), California indica (also referred to as “CL”), Cannatonic (also referred to as “CN”), Amazing Haze (also referred to as “AH”) Michka (also referred to as “MI”), Sensi 741 (also referred to as “741” and Afghani (also referred to as “AF”). The invention may notably be performed using the following Cannabis sativa cultivars: Jamaican Pearl, California indica, Cannatonic and Amazing Haze. All these cultivars are commercially available for example from Sensi Seeds, Amsterdam (www.sensiseeds.com).

The plant material inoculated in a temporary liquid immersion culture system in step a) is some plant material that may be obtained in one of the following manners:
Directly from various plant parts, including cells, stems, leaves, apical meristems, nodal segments, trichomes, inflorescence organs (such as sepals, calyx, stigmas and sugar leaves), or roots. Such directly obtained plant parts are referred to as explants.
Among such explants, those from apical meristem and/or nodal segments are particularly preferred. A mixture of explants from apical meristem and nodal segments may particularly be used.
Through differentiation of calli, using plant hormones, preferably cytokinins (see below in section relating to culture medium of step a)); or
Through de-differentiation or re-differentiation, growth and amplification of former explants.

Preferably, apical meristems and nodal segments from surface-sterilized seeds germinated in vitro will be used for inoculating temporary liquid immersion culture systems in step a).

Except for the addition in the temporary liquid immersion culture system of one or more bacterial and/or yeast strain(s), the rest of the method is preferably performed under sterile conditions, so that the explant inoculated into the temporary liquid immersion culture system in step a) should preferably have been sterilized before inoculation when directly obtained from a non-sterile plant part.

Sterilization may be performed by any appropriate method known to those skilled in the art, including washing with ethanol and bleach (a solution with 70-96% ethanol and a solution of bleach containing 0.1 to 10% active chlorine may notably be used), other methods, known by the man of the art, include the use of HgCl, Cl₂, Ozone, detergents and surfactants, fungicides and bactericides, or acids.

Alternatively, the explant may be inoculated directly into the temporary liquid immersion culture system in step a) when it has been obtained from plant material already cultured in sterile conditions.

### Temporary Liquid Immersion Culture System

The temporary liquid immersion culture system used in the method for producing Cannabis sativa cultivars plant tissues according to the invention is preferably a temporary immersion bioreactor (TIB).

TIB are self-contained sterile environments, in which the plant material is temporary immersed in liquid culture medium. Many TIB are available to those skilled in the art, as disclosed for instance in Etienne and Berthouly, 2002 (Plant Cell Tissue Organ Cult. 69, 215-231) and Watt, 2012 (Afr. J. Biotechnol. 11, 14025-14035; see notably Table 1 and references disclosed therein). Non-limiting examples of temporary immersion bioreactors include tilting and rocking vessels, twin flask systems (such as the BIT® system), or single containers with at least two compartments (such as Recipient for Automated Temporary Immersion (RITA®, WO96/25484), MATIS® (WO2012146872A1), or modified Nagene® polysulfone filtration system of NalgeNunc international), the Bioreactor of Immersion by Bubbles (BIB®). Other TIBs are for instance disclosed in WO2016092098, WO2019006466 WO2019006470, and M. Welander, J. et al. (Scientia Horticulturae, Volume 179, 2014, Pages 227-232), which are herein incorporated by reference in their entirety.

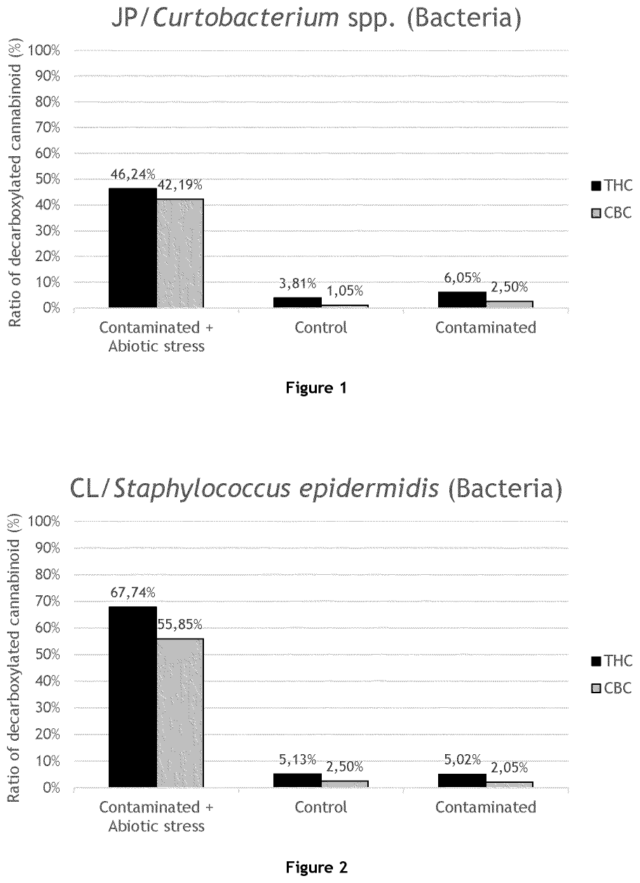

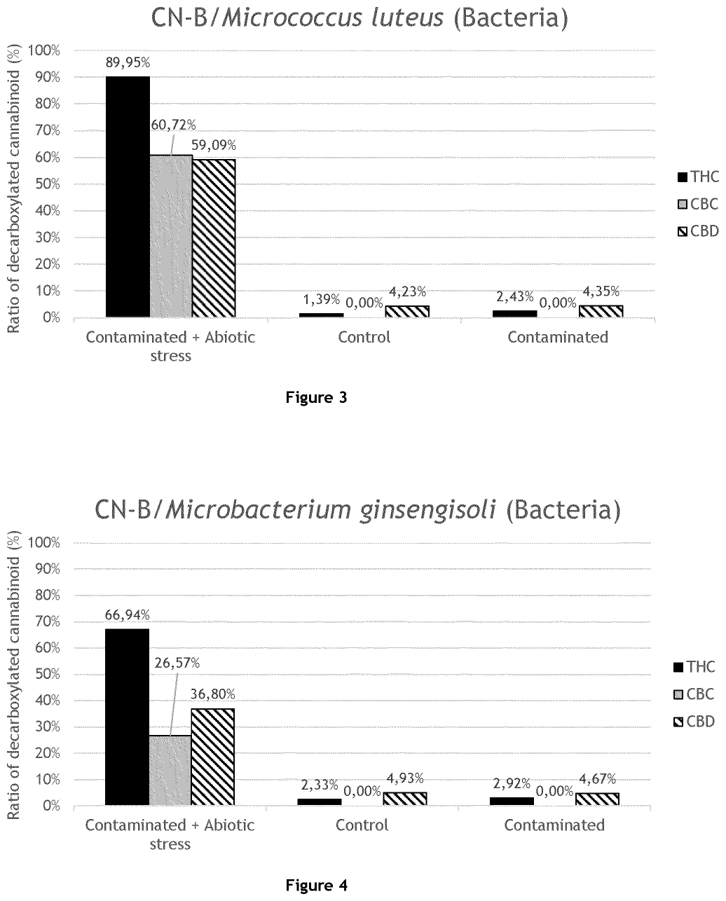

Preferred TIB for use in the invention include:
the Recipient for Automated Temporary Immersion (RITA®), a TIB in which the upper container containing the plant is linked to the lower compartment containing the medium and internal pressure regulates the movement of medium up or down in such a way that the immersion of cultures can be timed (Alvard et al., 1993. Plant Cell, Tissue and Organ Culture, 32, 55-60; WO96/25484). RITA® TIB are commercially available, for instance from VITROPIC, ZAE Des Avants, 34270 Saint-Mathieu-de-Treviers, FRANCE).
The MATIS®, a TIB using a technology comparable to RITA®, designed for higher culture volumes (WO2012146872A1). MATIS® TIB are commercially available, for instance from CID Plastiques, 50 rue de l'Oliveraie, ZAC Les Jasses, 34130 VALERGUES, France.
The Plantform, a TIB using a technology also comparable to RITA®, designed for higher culture volumes (Welander, J. et al. Scientia Horticulturae, Volume 179, 2014, Pages 227-232). Plantform TIB are commercially available, for instance from https://www.plantform.se.
The type of TIB disclosed in WO2016092098, which is based on the principle of a porous solid substrate upon which the cells reside that is immersed in liquid growth medium for short periods of time, and comprises (see in particular FIGS. 1-8, and their description):
   a growth chamber having one or more transparent side walls and a mesh bottom, to receive plant material;
   a flexible bag formed from a transparent material; the flexible bag having dimensioned to receive the growth chamber together with a liquid medium;
   an outer chamber having one or more transparent side walls, the outer chamber being formed to receive the growth chamber within the flexible bag.
   a driving mechanism arranged to selectively drive movement of the growth chamber along a single axis, allowing the immersion of the mesh bottom and the plant material during operation. Various driving mechanisms may be used, as disclosed in FIGS. 3 and 7-8 of WO2016092098.

No matter which driving mechanism is used, it preferably includes a timer to control operation of the driving mechanism and thereby, in use, control movement of the growth chamber between the first and second positions, thereby controlling the duration and frequency of immersion.

No matter which type of TIB is used in the method for producing Cannabis sativa cultivars plant tissues according to the invention, the volume of culture medium in said TIB is preferably from 1 to 10 000 L, preferably from 1 to 5 000 L, from 1 to 1 000 L, from 1 to 500 L, more preferably from 1 to 300 L, from 1 to 100 L, from 1 to 50 L, or from 1 to 10 L, depending on the desired scale-up for the production.

### Sterile Liquid Culture Medium

The temporary liquid immersion culture system in which the sterile plant material is inoculated in step a) contains a sterile liquid culture medium containing at least one cytokinin and/or at least one auxin.

The sterile liquid culture medium is based on a sterile basal culture medium containing all nutrients necessary for growth, including:
a solution of salts supplying the macro and micro-elements necessary for the growth of whole plants,
a carbon source (usually sucrose); and
optionally, various vitamins, various amino acids and/or various undefined supplements (such as extracts from yeast, meat, malt, various fruits and plants, protein hydrolysates).

Any suitable basal culture medium suitable for plant propagation in vitro may be used, such as the following commercially available media: MS (Murashige and Skoog) medium, DKW medium (Driver and Kuniyaki Walnut medium, Driver J A, Kuniyuki A H, 1984, In vitro propagation of Paradox walnut rootstock. Hortscience 19: 507-509), B5 (Gamborg) medium, WPM (Woody Plant Medium) medium, or SH (Schenk and Hildebrandt) medium, preferably MS, DKW, SH or B5 medium, more preferably MS or DKW medium. All of these media are commercially available, for instance from Sigma Aldrich.

The concentration at which the basal culture medium is used is typically 2×, 1× or 0.5×.

Addition of macroelements (N, P, K) can be considered to further optimize growth and metabolism.

The carbon source can be selected from sugars such as glucose, fructose or more commonly sucrose. Sugar concentration in the culture medium is preferably in the range from 10 to 60 g/l (corresponding to 1 to 6% sugar, preferably sucrose), preferably from 15 to 40 g/L (i.e. 1.5 to 4%), from 20 to 40 g/L (i.e. 2 to 4%), from 25 to 35 g/L (i.e. 2.5 to 3.5%), usually around 30 g/L (i.e. 3%). High sugar concentration in the culture medium may cause osmotic stress, having potential detrimental effect on growth but potentially inducing cannabinoids biosynthesis. In contrast, low sugar concentration in the culture medium may also limit growth due to insufficient energy available to the plants.

Vitamins are preferably present in the liquid culture medium, and may be selected from the following commercially available vitamins: MS vitamins, DKW vitamins, B5 vitamins, WPM vitamins, SH vitamins, preferably used in conjunction with the corresponding basal culture medium. Addition or suppression of vitamins and microelements can be considered to further optimize growth and metabolism.

The sterile liquid culture medium also contains at least one cytokinin and/or at least one auxin. “Cytokinins” are a group of chemicals that primarily influence cell division and shoot formation but also have roles in delaying cell senescence, are responsible for mediating auxin transport throughout the plant, and affect internodal length and leaf growth. “Auxins” are compounds that positively influence cell enlargement, bud formation and root initiation. They also promote the production of other hormones and in conjunction with cytokinins, they control the growth of stems, roots, fruits and are involved in flowering.

In the method for producing plant tissues containing one or more decarboxylated cannabinoid(s) according to the invention, a natural or artificial cytokinin belonging to the adenine-type or the phenylurea-type is preferably used, more preferably said cytokinin is selected from adenine, kinetin, zeatin, 6-benzylaminopurine, diphenylurea (DPU), N-(2-chloro-4-pyridyl)-N-phenylurea (CPPU), thidiazuron (TDZ) and derivatives thereof having cytokinin activity, more preferably said cytokinin is selected from thidiazuron (TDZ), Benzylaminopurine (BAP), Meta-Topolin (mT), Kinetin (Kin), DPU or CPPU, most preferably from thidiazuron (TDZ), Benzylaminopurine (BAP), Meta-Topolin (mT), or Kinetin (Kin).

Based on common general knowledge, a skilled person will know which cytokinin(s) to select, depending on the origin (explant type and part of plant from which it is directly or indirectly derived) of inoculated plant material.

For Cannabis sativa cultivars, preferred cytokinins comprised in the sterile culture medium in step a) are selected from thidiazuron (TDZ), Benzylaminopurine (BAP), Meta-Topolin (mT), or Kinetin (Kin), in particular said cytokinin(s) is/are thidiazuron (TDZ) and/or Meta-Topolin (mT).

With respect to cytokinin concentration (when present), the sterile liquid culture medium in the temporary liquid immersion culture system preferably comprises from 0.01 to 10 mg/L, preferably from 0.1 to 5 mg/L of said cytokinin.

In the method for producing plant tissues containing one or more decarboxylated cannabinoid(s) according to the invention, the sterile liquid culture medium may alternatively or further comprise at least one auxin, as defined above. Appropriate auxins for use in the invention include naturally occurring auxins, such as 4-chloro-indoleacetic acid, phenylacetic acid (PAA), indole-3-butyric acid (IBA) and indole-3-acetic acid (IAA); or synthetic auxin analogues, such as 1-naphthaleneacetic acid (NAA), and 2,4-dichlorophenoxyacetic acid (2,4-D).

When present, the concentration of auxin in the sterile liquid culture medium is from 0.01 to 10 mg/L, more preferably between 0.1 and 5 mg/L.

The sterile liquid culture medium in the temporary liquid immersion culture system may contain at least one cytokinin (without any auxin), at least one auxin (without any cytokinin), or at least one cytokinin and at least one auxin.

### Inoculation

Inoculation is made by the more appropriate method, depending on the type of temporary liquid immersion culture system used.

Inoculum size typically ranges from 1 to 50 explants, preferably 1 to 20 explants or even 5 to 15 explants (in particular explants from nodal segments and apical meristems), per 100 ml of sterile culture medium. Usually, about 10 explants are used to inoculate 100 ml medium (Watt, 2012. Afr. J. Biotechnol. 11, 14025-14035).

Step b)—Cultivation for Growing of Plant Biomass

In step b), the plant material is cultivated in the temporary liquid immersion culture system under conditions suitable for growing plant biomass.

“Plant biomass” refers to all plant tissues present in the temporary liquid immersion culture system, such as leaves, stems, roots, flowers. . . .

Preferably, mainly leafy biomass is grown during step b), although other plant tissues may also grow. “Leafy biomass” is an expression widely used in the domain of plant growing, and refers to plant material composed mainly of leaf tissue and stems, without excluding the occasional minor presence of other organs such as roots or flowers. Leaf tissue is distinguished from other plant tissues by their shape, their higher number of chloroplasts and developing chloroplasts (as counted by confocal microscopy analysis), and the higher photosynthetic activity (determination of Fv/Fm with fluorometer) and chlorophyll content (by analysis of extracted pigments by absorption spectrophotometry) of chloroplasts, as detected by the absorption of carbon dioxide by the plant tissue. Such methods of determination are well known to the skilled person as for example as described in (Baker (2008) Ann. Rev. Plant Biol. 59: 89-113). When referring to leafy biomass, it is intended to refer to plant material comprising at least 50%, preferably 70%, and more preferably greater than 85% leaf tissue.

### Sterile Liquid Culture Medium

The sterile liquid culture medium used in step b) for growing plant biomass may or not have the same composition as the sterile liquid culture medium used in step a).

Preferably, the same basal culture medium as in step a) is used in step b), in order not to have to remove the culture medium from the temporary liquid immersion culture system. The sterile liquid culture medium used in step b) also comprises at least one cytokinin and/or at least one auxin, as defined above for the culture medium of step a).

However, various compounds may further be added in the sterile liquid culture medium of step b).

For instance, additional amounts of a sterile carbon source (preferably sucrose) may be added during step b), in order to ensure that sufficient carbon and energy supply is present in the culture medium during the whole duration of step b). When performed, addition of additional amounts of a sterile carbon source (preferably a sucrose solution) during step b) is performed under sterile conditions to ensure that the produced plant tissue remains sterile.

Sterile phytohormones (such as cytokinins, auxins or other phytohormones, more preferably cytokinins and/or auxins, where said cytokinins are selected from adenine, kinetin, zeatin, 6-benzylaminopurine, diphenylurea, thidiazuron (TDZ) and derivatives thereof having cytokinin activity and said auxins are selected from naturally occurring auxins, such as 4-chloro-indoleacetic acid, phenylacetic acid (PAA), indole-3-butyric acid (IBA) and indole-3-acetic acid (IAA); or synthetic auxin analogues, such as 1-naphthaleneacetic acid (NAA), and 2,4-dichlorophenoxyacetic acid (2,4-D)) may also be added during step b) to compensate hormone degradation over time or to further optimize growth by inducing physiological changes in the plant material, notably affecting the development of axillary buds, development of leaves, and/or elongation of stems.

### Duration and Frequency of Immersion

The “duration of immersion” is defined as the duration during which the plant material is immersed in the culture medium.

The “frequency of immersion” is defined as follows:

Frequency of immersion=1/duration in hours between two immersions of the plant material in the culture medium.

The frequency of immersion decreases when the duration between two immersions of the plant material in the culture medium increases. However, for practical reasons, in the following the frequency of immersion will in fact be expressed as a duration (in minutes or hours) between the starting time of two consecutive immersions of the plant material in the culture medium.

In step b) of growing plant biomass, the duration and frequency of immersion of the plant material in the culture medium preferably varies from 30 seconds to 15 minutes every 30 minutes to 12 hours.

Depending on the origin (explant type and part of plant from which it is directly or indirectly derived) of plant material inoculated in step a), a skilled person will be able to optimize the duration and frequency of immersion of the plant material in the culture medium in step b) in order to favor the growing of plant biomass.

In step b), a duration and frequency of immersion of the plant material in the culture medium from 1 to 10 minutes every 2 to 8 hours, more preferably from 1 to 5 minutes every 4 to 8 hours will generally be appropriate (in particular in the RITA® or Plantform TIB). Preferably, a duration and frequency of immersion of the plant material in the culture medium from 2 to 4 minutes every 4 to 8 hours, more preferably from 2.5 to 3.5 minutes every 5 to 7 hours, most preferably 3 minutes every 6 hours may be used in step b).

However, other appropriate durations and frequencies may be determined by a skilled person based on common general knowledge about the plant material of interest. The goal of the duration and frequency of immersion is to ensure the plant material maintains a degree of humidity that is favorable for growing plant biomass.

Therefore, while the above-mentioned durations and frequencies are examples of appropriate durations and frequencies, alternative appropriate (duration/frequency) combinations, in which duration is decreased and frequency increased or duration is increased and frequency is decreased, may be determined by a skilled artisan.

In particular, for optimization of the duration and frequency of immersion in step b) of growing plant biomass, various durations and frequencies may be tested and the weight of total plant biomass produced (i.e. the total yield) and plant material quality monitored to identify the best immersion conditions.

### Light Conditions

Depending on the origin (explant type and part of plant from which it is directly or indirectly derived) of plant material inoculated in step a), a skilled person will know which appropriate light conditions for growing plant biomass should be used in step b), based on common general knowledge about the plant material of interest.

Relevant light parameters that may be optimized include:
photoperiod,
light type (wavelength notably), and
light intensity.

“Photoperiod” refers to the time that a plant or animal is exposed to light over a given period, usually over 24-hours. In step b), an appropriate photoperiod may be a regimen of 12 to 24 hours of light and 0 to 12 hours of darkness, preferably 12 to 18 hours of light and 6 to 12 hours of darkness, 14 to 18 hours of light and 6 to 10 hours of darkness, such as 16 hours of light and 8 hours of darkness.

Type of light: light can be supplied as white light which includes the entire spectrum, as red (−625-700 nm) and blue (−450-520 nm) lights (corresponding to the absorption peaks of chlorophyll), or as a mix thereof. Other wavelengths might be involved in growth and tissue differentiation, such as flowering or flower maturation, and might thus be added.

Light intensity is preferably in ranges from 50-600 μmol photons/m²/s of Photosynthetically Active Radiations (PAR), preferably 100-300 μmol photons/m²/s.

### Other Culture Parameters

Depending on the origin (explant type and part of plant from which it is directly or indirectly derived) of plant material inoculated in step a), further culture parameters may be optimized by a skilled person for growing plant biomass, based on common general knowledge about the specific Cannabis sativa cultivar plant material used.

These may notably include:
Temperature:
Temperature may be optimized between 18 and 35° C. (more preferably between 23 and 30° C., such as about 25, 26 or 27° C.) in the case of Cannabis sativa cultivars (Potter, 2014. Drug Test. Anal. 6, 31-38).
Gas composition:
Gas composition is to be controlled and can be optimized for growth and production by enriching the air in either CO₂ or O₂. For Cannabis sativa cultivars, a CO₂ concentration of 0.05 to 5%, and an O₂ concentration of 5 to 50% are particularly suitable.

For Cannabis sativa cultivars, a temperature between 18 and 35° C. (more preferably between 23 and 30° C., such as about 25, 26 or 27° C.), a CO₂ concentration of 0.05 to 5%, and an O₂ concentration of 5 to 50% will preferably use used in step b).

### Duration of Step b)

The duration of step b) is selected so that a maximum of vegetative growth can be obtained before the senescence of the plant tissue and may depend on whether step c) is performed before or after step b) (see below section “Timing of step c) and durations of steps b) and d)”).

Step c)—Inoculation with One or More Bacterial and/or Yeast Strain(s)

In step c), the sterile liquid culture medium is inoculated with one or more bacterial and/or yeast strain(s).

Bacterial and/or Yeast Strain(s)

Experimental data provided in examples below show that inoculation of the sterile liquid culture medium with several types of bacterial strains and also yeasts may permit to obtain plant tissues containing decarboxylated cannabinoids (in particular THC, CBC, CBD, or any combination thereof).

In addition, the bacterial strains able to generate plant tissues containing decarboxylated cannabinoids (in particular THC, CBC, CBD, or any combination thereof) when inoculated in the sterile liquid culture medium are quite diverse (see Table 1 below) and it may thus be expected that the use of any bacterial strain and/or yeast strain for inoculation of the sterile liquid culture medium in step c) would lead to a similar result. Any bacterial strain, any yeast strain, any combination of bacterial strains, any combination of yeast strains, or any combination of bacterial and yeast strains may thus be used in step c) for inoculation of the sterile liquid culture medium.

However, although any bacterial or yeast strain may be expected to permit to naturally obtain plant tissues containing a significant amount and proportion of decarboxylated cannabinoids when inoculated into the sterile liquid culture medium in step c), some bacterial and yeast strains may be more suitable than others in the method for producing plant tissues containing one or more decarboxylated cannabinoid(s) according to the invention. In particular, to be adapted for the co-culture with the plants in the claimed method, selected bacterial and/or yeast strains should preferably grow in presence of light (preferably in ranges from 50-600 μmol photons/m²/s of Photosynthetically Active Radiations (PAR), preferably 100-300 μmol photons/m²/s) and air (bacterial or yeast species should preferably be strictly or facultatively aerobic), at a temperature between 18 and 35° C. (more preferably between 23 and 30° C., such as about 25, 26 or 27° C.)

Moreover, based on bacterial and yeast strains shown as useful in the method for producing plant tissues containing one or more decarboxylated cannabinoid(s) according to the invention in experimental data presented in examples below, some specific bacterial and/or yeast strain(s) may preferably be used.

Bacterial and yeast strains found by the inventors as useful for the method for producing plant tissues containing one or more decarboxylated cannabinoid(s) according to the invention are presented in Table 1 below.

TABLE 1
Information about bacterial and yeast strains found to be useful in the method for producing sterile
plant tissues containing one or more decarboxylated cannabinoid(s) according to the invention.

| Bacteria/ | | | | | |
|---|---|---|---|---|---|
| yeast | Species | Genus | Family | Order | Class |
| Bacteria | Not defined | Curtobacterium | Microbacteriaceae | Micrococcales | Actinomycetia |
| Bacteria | Microbacterium | Microbacterium | Microbacteriaceae | Micrococcales | Actinomycetia |
| | ginsengisoli | | | | |
| Bacteria | Micrococcus | Micrococcus | Micrococcaceae | Micrococcales | Actinomycetia |
| | luteus | | | | |
| Bacteria | Staphylococcus | Staphylococcus | Staphylococcaceae | Bacillales | Bacilli |
| | epidermidis | | | | |
| Bacteria | Bacillus | Bacillus | Bacillaceae | Bacillales | Bacilli |
| | megaterium | | | | |
| Bacteria | Roseomonas | Roseomonas | Acetobacteraceae | Rhodospirillales | Alphaproteobacteria |
| | mucosa | | | | |
| Bacteria | Sphingomonas | Sphingomonas | Sphingomonadaceae | Sphingomonadales | Alphaproteobacteria |
| | paucimobilis | | | | |
| Yeast | Cystobasidium | Cystobasidium | Cystobasidiaceae | Cystobasidiales | Cystobasidiomycetes |
| | minutum | | | | |

As a result, when one or more bacterial strain(s) is(are) inoculated in step c), at least one bacterial strain(s) is(are) preferably selected from:
a) The Actinomycetia, Alphaproteobacteria, Bacilli classes;
b) The Micrococcales, Rhodospirillales, Bacillales, and Sphingomonadales orders, more preferably the Micrococcales, Rhodospirillales and Bacillales orders;
c) The Microbacteriaceae, Micrococcaceae, Acetobacteraceae, Staphylococcaceae, Sphingomonadaceae and Bacillaceae families, more preferably the Microbacteriaceae, Micrococcaceae, Acetobacteraceae, and Staphylococcaceae families;
d) The Curtobacterium, Microbacterium, Micrococcus, Roseomonas, Staphylococcus, Sphingomonas and Bacillus genus, more preferably the Curtobacterium, Microbacterium, Micrococcus, Roseomonas, and Staphylococcus genus; or
e) The Curtobacterium genus, Microbacterium ginsengisoli, Micrococcus luteus, Roseomonas mucosa, Staphylococcus epidermidis, Sphingomonas paucimobilis and Bacillus megaterium species, more preferably the Curtobacterium genus, Microbacterium ginsengisoli, Micrococcus luteus, Roseomonas mucosa and Staphylococcus epidermidis species.

Similarly, based on experimental data presented in examples below, when one or more yeast strain(s) is(are) inoculated in step c), at least one yeast strain(s) is(are) preferably selected from the Cystobasidiomycetes class, the Cystobasidiales order, the Cystobasidiaceae family, the Cystobasidium genus, or the Cystobasidium minutum species.

Preparation of the One or More Bacterial and/or Yeast Strain(s)

The one or more bacterial and/or yeast strain(s) can be prepared in any suitable sterile (before bacteria and/or yeasts addition) basal culture medium suitable for bacterial propagation (including but not limited to LB (Lysogeny Broth), TSB (Tryptic Soy Broth) . . . ) or any plant propagation medium (as disclosed above in section relating to the sterile liquid culture medium).

Preferably, the one or more bacterial and/or yeast strain(s) inoculated in step c) are preferably in exponential growth phase, thus ensuring their viability during the co-culture. For instance, before inoculation, the one or more bacterial and/or yeast strain(s) may have been cultured for 1 to 10 days, preferably 2 to 8 days, 2 to 6 days or 3 to 5 days, such as about 4 days, in sterile culture medium suitable for bacterial propagation, including but not limited to LB, TSB, DKW, MS, preferably TSB or DKW.

Concentration of Bacterial and/or Yeast Strain(s) Inoculated in Step c)

Any appropriate concentration of bacterial and/or yeast strain(s) may be inoculated into the sterile liquid culture medium in step c).

The appropriate concentration may depend on the timing of step c) and the durations of steps b) and d). In particular, if a too low concentration of bacterial and/or yeast strain(s) is added or if bacterial and/or yeast strain(s) are added too close to senescence of the plant tissues, the decarboxylation yield will not be optimal. In contrast, if a too high concentration of bacterial and/or yeast strain(s) is used or if bacterial and/or yeast strain(s) are added too early during vegetative growth, then it is the total cannabinoids yield (decarboxylated or not) that will not be optimal.

Therefore, in order to optimize total cannabinoids yield and decarboxylation yield, the inoculated concentration of bacterial and/or yeast strain(s), the timing of step c) and the durations of steps b) and d) should be optimized, based on common general knowledge in the field.

A suitable concentration of bacterial and/or yeast strain(s) in the temporary liquid immersion culture system (preferably TIB) (after inoculation) may be selected in the range of an optical density (OD) at 600 nm in the temporary liquid immersion culture system (preferably TIB) between 0.0001 and 0.1.

The OD at 600 nm may be measured using any conventional technique, for instance, UVisco Spectrophotometer V-1100D.

Step d)—Cultivation in the Presence of One or More Bacterial and/or Yeast Strain(s)

In step d), the plant material in the temporary liquid immersion culture system inoculated by the bacterial and/or yeast strain(s) is cultivated, optionally under abiotic stress, in order to further grow plant biomass and decarboxylate the cannabinoids inside the plant tissues.

### Liquid Co Culture Medium

Except for the inoculated one or more bacterial and/or yeast strain(s), the liquid co culture medium used in step d) is still made of sterile components, in order to ensure that only the selected bacterial and/or yeast strain(s) inoculated in step c) are present in the medium. In other words, even if the liquid culture medium is no more sterile due to the presence of the inoculated one or more bacterial and/or yeast strain(s), the culture preferably remains a controlled culture, without other microorganisms than those inoculated in step c).

The liquid co-culture medium used in step d) may or not have the same composition as the sterile liquid culture media used in steps a) and b).

Preferably, the same basal culture medium as in steps a) and b) is used in step d), in order not to have to remove the culture medium from the temporary liquid immersion culture system.

The sterile liquid culture medium used in step d) also comprises at least one cytokinin and/or at least one auxin, as defined above for the culture medium of step a).

However, various compounds may further be added in the sterile liquid culture medium of step d).

For instance, additional amounts of a sterile carbon source (preferably sucrose) may be added during step d), in order to ensure that sufficient carbon and energy supply is present in the sterile culture medium during the whole duration of step d). When performed, addition of additional amounts of a sterile carbon source (preferably a sucrose solution) during step d) is performed under sterile conditions to ensure that the produced plant tissue remains sterile, excepted for the presence of the one or more bacterial and/or yeast strain(s) inoculated in step c).

Sterile phytohormones (such as cytokinins, auxins or other phytohormones, more preferably cytokinins and/or auxins, where said cytokinins are selected from adenine, kinetin, zeatin, 6-benzylaminopurine, diphenylurea, thidiazuron (TDZ) and derivatives thereof having cytokinin activity and said auxins are selected from naturally occurring auxins, such as 4-chloro-indoleacetic acid, phenylacetic acid (PAA), indole-3-butyric acid (IBA) and indole-3-acetic acid (IAA); or synthetic auxin analogues, such as 1-naphthaleneacetic acid (NAA), and 2,4-dichlorophenoxyacetic acid (2,4-D)) may also be added during step d) to compensate hormone degradation over time or to further optimize growth by inducing physiological changes in the plant material, notably affecting the development of axillary buds, development of leaves, and/or elongation of stems.

### Other Culture Parameters and Optional Abiotic Stress

Other culture parameters (duration and frequency of immersion, light conditions, temperature, gas composition, etc.) may or not be the same as in step b).

In addition, other culture parameters (duration and frequency of immersion, light conditions, temperature, gas composition, etc.) may or not be the same during all of step d), or may vary during step d).

In particular, step d) may be performed under abiotic stress, as data provided in examples below show that this enhances the rate of cannabinoids decarboxylation.

By “abiotic stress”, it is referred to a significant negative impact of non-living factors on plant population performance or individual plant physiology in a given environment. In the context of culture in a temporary liquid immersion culture system, “abiotic stress” more particularly refers to significant negative impact of non-living factors on plant population growth or individual plant physiology in the temporary liquid immersion culture system.

Culture parameters of the temporary liquid immersion culture system that may be changed in order to induce abiotic stress include:
Duration and frequency of immersion:
In particular, drought is known to induce abiotic stress. Abiotic stress may thus be induced by decreasing the duration and/or the frequency of immersion to values that alter plant population growth or individual plant physiology.
Depending on the specific Cannabis sativa cultivar and on the specific temporary liquid immersion culture system used and based on his/her common general knowledge, skilled person will know to which values the duration and/or the frequency of immersion should be decreased to induce abiotic stress.
Moreover, the ability of specific duration/frequency of immersion couples to induce abiotic stress may be easily assessed for any Cannabis sativa cultivar and temporary liquid immersion culture system by applying varying duration/frequency of immersion couples and assessing the loss of water in the biomass due to evaporation during drought (e.g. by weighing plants before and after decreasing the duration and/or the frequency of immersion), or other biomarkers of plant stress (e.g. accumulation of plant hormones, of oxygen reactive species, induction of genes known to be associated to stress response).
In particular, for most Cannabis sativa cultivar cultivated in the RITA® or Plantform TIB, a duration and frequency of immersion of the plant material in the culture medium from 1 to 10 minutes every 2 to 8 hours, more preferably from 1 to 5 minutes every 4 to 8 hours will generally be appropriate for maintaining plant physiology. On this basis, abiotic stress may be induced by:
   decreasing the duration of immersion to a value between 0 and 1 minute; and/or
   decreasing the frequency of immersion to a value between 24 hours and 3 weeks.
In particular, abiotic stress may be induced by completely stopping immersion in the temporary liquid immersion culture system (preferably TIB).
Light conditions:
Insufficient or excess level of light are also known to induce abiotic stress. Abiotic stress may thus be induced by decreasing or increasing light photoperiod and/or light intensity to values that alter plant population growth or individual plant physiology.
Depending on the specific Cannabis sativa cultivar and on the specific temporary liquid immersion culture system used and based on his/her common general knowledge, skilled person will know to which values the light photoperiod and/or light intensity should be decreased or increased to induce abiotic stress.
Moreover, the ability of specific light photoperiod/intensity couples to induce abiotic stress may be easily assessed for any Cannabis sativa cultivar and temporary liquid immersion culture system by applying varying light photoperiod/intensity couples and assessing the impact on plant development by comparing the same Cannabis sativa cultivar in conditions known to maintain plant physiology and in conditions with decreased or increased light photoperiod and/or light intensity and assessing plant development parameters, including but not limited to: growth arrest, leaf size, plant etiolation, and leaf color (loss of chlorophyll with a deficit of light gives yellowing, and accumulation of pigment in response to high level of light gives darkening and browning). Preferably, such comparison is made at the beginning of culture in the temporary liquid immersion culture system, in order to prevent an effect of plant senescence on the assessed plant development parameters.
Temperature:
High or low temperatures are known to induce abiotic stress. Abiotic stress may thus be induced by decreasing or increasing temperature in the temporary liquid immersion culture system to lower or higher values that alter plant population growth or individual plant physiology.
Depending on the specific Cannabis sativa cultivar and on the specific temporary liquid immersion culture system used and based on his/her common general knowledge, skilled person will know to which values the temperature should be decreased or increased to induce abiotic stress.
Moreover, the ability of specific temperatures to induce abiotic stress may be easily assessed for any Cannabis sativa cultivar and temporary liquid immersion culture system by applying varying temperatures and assessing the impact on plant development parameters (see above for light conditions). Here also, such comparison is preferably made at the beginning of culture in the temporary liquid immersion culture system, in order to prevent an effect of plant senescence on the assessed plant development parameters.

Preferably, when step d) is under abiotic stress, abiotic stress is thus preferably induced by:
decreasing the duration and/or the frequency of immersion to values that alter plant population growth or individual plant physiology;
decreasing or increasing light photoperiod and/or light intensity to values that alter plant population growth or individual plant physiology; and/or
decreasing or increasing temperature in the temporary liquid immersion culture system to lower or higher values that alter plant population growth or individual plant physiology.

More preferably, when step d) is under abiotic stress, abiotic stress is thus preferably induced by decreasing the duration and/or the frequency of immersion (any embodiment described above).

Step d) is said to be performed “under abiotic stress” when it is performed partially or entirely under abiotic stress.

Step d) is said to be performed “partially under abiotic stress” when part of the duration of step d) is performed under culture conditions inducing abiotic stress. The part performed under culture conditions inducing abiotic stress may be at the beginning, in the middle or at the end of step d), preferably it is at the end of step d).

Step d) is said to be performed “entirely under abiotic stress” when the whole duration of step d) is performed under culture conditions inducing abiotic stress.

Alternatively, step d) may also be performed without abiotic stress (i.e. only culture parameters not inducing abiotic stress are used during the whole duration of step d)), as examples show that decarboxylation of cannabinoids may occur without abiotic stress (see Example 2 below).

The duration of step d) is selected so that a maximum of non-acidic forms of cannabinoids can be obtained as quickly as possible without negative impact on the total production of cannabinoids and plant biomass, and may depend on whether step c) is performed before or after step b) (see below section “Timing of step c) and durations of steps b) and d)”).

### Timing of Step c) and Durations of Steps b) and d)

Step c) of inoculating the sterile liquid culture medium with one or more bacterial and/or yeast strain(s) is necessarily performed before step d) (as step d) involves culture in the presence of the inoculated one or more bacterial and/or yeast strain(s)), but may be performed before or after step b).

When step c) is performed after step b) (first embodiment), the plant material is first cultivated in the temporary liquid immersion culture system under conditions suitable for growing plant biomass under sterile conditions, then one or more bacterial and/or yeast strain(s) are inoculated in the sterile liquid culture medium, and the plant material is further cultivated in the presence of the inoculated one or more bacterial and/or yeast strain(s), optionally under abiotic stress.

In this first embodiment, steps b) and d) are distinct steps, and only step d) is performed in the presence of the inoculated one or more bacterial and/or yeast strain(s). This may be advantageous as the presence of bacteria and/or yeasts may alter the growth of plant biomass (due to biotic stress), and thus the final yield in cannabinoids. Therefore, performing a step b) of growing plant biomass before inoculation of one or more bacterial and/or yeast strain(s)) may favor plant biomass yield, the cannabinoids of which are then decarboxylated during step d), after inoculation of one or more bacterial and/or yeast strain(s) in step c).

As most of plant biomass growth has already been obtained in step b), a concentration of bacterial and/or yeast strain(s) in the higher part of the general range described above (such as an optical density in the temporary liquid immersion culture system (preferably TIB) between 0.001 and 0.1, preferably between 0.005 and 0.1, more preferably between 0.01 and 0.05) may possibly be inoculated in step c), in order to increase the rate of cannabinoids decarboxylation.

In addition, also due to the fact that most of plant biomass growth has already been obtained in step b), step d) may then preferably be performed entirely under abiotic stress, as this is shown in examples below to enhance the rate of cannabinoids decarboxylation.

In this first embodiment, a suitable duration for step b) is about 2 to 6 weeks, in particular 2 to 4 weeks, such as about 3 weeks. This is particularly true in the case where a Cannabis sativa cultivar inoculated at a density of 1 to 20 (preferably 5 to 15, more preferably about 10) explants (in particular explants from apical meristems and nodal segments) per 100 ml of sterile culture medium in a temporary liquid immersion culture system (in particular a TIB, such as RITA® or Plantform TIB). In addition, a suitable duration for step d) is about 1 to 7 weeks, in particular 4 to 6 weeks.

When step c) is performed before step b) (second embodiment), one or more bacterial and/or yeast strain(s) are inoculated in the sterile liquid culture medium of the temporary liquid immersion culture system (before or after step a) of inoculating the sterile plant material), and culture of the plant material is then performed only in the presence of the inoculated one or more bacterial and/or yeast strain(s).

In this other embodiment, steps a) and c) are first performed (in any order, a) followed by c) or c) followed by a), and steps b) and d) are then performed simultaneously as a single step, after inoculation of one or more bacterial and/or yeast strain(s). This embodiment may favor rapid decarboxylation of cannabinoids just after their production in plant tissues.

However, as biotic stress due to the presence of one or more bacterial and/or yeast strain(s) may alter plant biomass growth and final yields in cannabinoids (see Example 2 below and FIG. 8), a concentration of bacterial and/or yeast strain(s) rather in the lower part of the general range described above (such as an optical density in the bioreactor between 0.0001 and 0.006, preferably between 0.003 and 0.006) is preferably inoculated in step c), in order to preserve plant biomass growth during combined steps b) and d).

Similarly, as abiotic stress may also alter plant biomass growth and final yields in cannabinoids, simultaneous steps b) and d) are preferably performed only partially under abiotic stress, also to preserve plant biomass growth during combined steps b) and d). Preferably, about 50 to 70% (preferably 55 to 60%) of the total duration of combined steps b) and d) is performed without abiotic stress, and about 30 to 50% (preferably 40 to 45%) of the total duration of combined steps b) and d) is performed under abiotic stress.

In this second embodiment, the total duration of combined steps b) and d) is preferably about 5 to 9 weeks, in particular 6 to 8 weeks, such as about 7 weeks. When simultaneous steps b) and d) are performed only partially under abiotic stress (which is preferred in this second embodiment), the duration without abiotic stress is preferably about 2 to 6 weeks, in particular 2 to 4 weeks, such as about 3 weeks, and the duration under abiotic stress is preferably about 1 to 5 weeks, in particular 3 to 4 weeks. Abiotic stress is preferably performed during 2 to 4 weeks at the end of simultaneous steps b) and d). This is particularly true in the case where a Cannabis sativa cultivar inoculated at a density of 1 to 10 (preferably 1 to 5, more preferably about 3) explants (in particular explants from apical meristems and nodal segments) per 100 ml of sterile culture medium in a temporary liquid immersion culture system (in particular a TIB, such as RITA® or Plantform TIB).

Preferred embodiments include:
i) successive and distinct steps b), c) and d), wherein:
   step b) is performed without abiotic stress during 2 to 4 weeks,
   the OD at 600 nm of the one or more bacterial and/or yeast strain(s) after inoculation in the temporary liquid immersion culture system is between 0.001 and 0.1 (preferably between 0.005 and 0.1, more preferably between 0.01 and 0.05),
   the duration of step d) is about 1 to 7 weeks, in particular 4 to 6 weeks, and
   step d) is performed partially under abiotic stress, wherein abiotic stress is added during 2 to 4 weeks at the end of step d).
ii) step c) followed by simultaneous steps b) and d), wherein:
   the OD at 600 nm of the one or more bacterial and/or yeast strain(s) after inoculation in the temporary liquid immersion culture system is between 0.0001 and 0.006 (preferably between 0.003 and 0.006),
   the duration of simultaneous steps b) and d) is about 5 to 9 weeks, in particular 6 to 8 weeks, and
   simultaneous steps b) and d) are performed partially under abiotic stress, wherein abiotic stress is added during 2 to 4 weeks at the end of simultaneous steps b) and d).

In preferred embodiments i) and ii) above, Cannabis sativa cultivar are preferably inoculated at a density of 1 to 20 (preferably 5 to 15, more preferably about 10) explants (in particular explants from apical meristems and nodal segments) per 100 ml of sterile culture medium in a temporary liquid immersion culture system (in particular a TIB, such as RITA® or Plantform TIB) in step a).

Step e)—Collection of Plant Tissues Containing One or More Decarboxylated Cannabinoid(s)

In step e), plant tissues containing one or more decarboxylated cannabinoid(s) are collected.

This may be done by standard methods known in the art. These methods include but are not limited to emptying of the bioreactor chamber containing the plant material, separating the biomass from the vessel and collection in appropriate container for direct drying, freeze drying or direct extraction on fresh material.

Step e) may further comprise collecting the culture medium, as some cannabinoids may be present in the culture medium and may thus be extracted from the culture medium.

### Method for Producing One or More Decarboxylated Cannabinoid(s)

The present invention also relates to a method for producing one or more decarboxylated cannabinoid(s), the method comprising:
a) producing plant tissues containing one or more decarboxylated cannabinoid(s) using the method for producing plant tissues containing one or more decarboxylated cannabinoid(s) according to the invention,
b) collecting or extracting the one or more decarboxylated cannabinoid(s) from the obtained plant tissues, and optionally from the culture medium of the temporary liquid immersion culture system,
c) optionally, purifying the collected or extracted one or more decarboxylated cannabinoid(s), and
d) optionally, adding an acceptable diluent, excipient or carrier to the one or more decarboxylated cannabinoid(s).
   Step a)—Production of Plant Tissues Containing One or More Decarboxylated Cannabinoid(s)

Step a) of the method for producing one or more decarboxylated cannabinoid(s) according to the invention is performed as described above for the method for producing plant tissues containing one or more decarboxylated cannabinoid(s) according to the invention. In particular, any general or preferred embodiment disclosed above for the method for producing plant tissues containing one or more decarboxylated cannabinoid(s) according to the invention also applies to step a) of the method for producing one or more decarboxylated cannabinoid(s) according to the invention.

Step b)—Collection or Extraction of the One or More Decarboxylated Cannabinoid(s) from the Obtained Plant Tissues, and Optionally from the Culture Medium of the Temporary Liquid Immersion Culture System

In step b), the one or more decarboxylated cannabinoid(s) present in the obtained plant tissues, and optionally in the culture medium of the temporary liquid immersion culture system, are collected or extracted.

For cannabinoids that are released or secreted outside of the plant tissues, they may be directly extracted from the culture medium of the temporary liquid immersion culture system used in step a), without a need for extraction from plant tissues.

Other cannabinoids are extracted from the plant tissues (stem, leaves, and optionally flowers) according to standard methods known in the art. These methods include but are not limited to drying the plant tissues (stem, leaves, and optionally flowers) and crushing it, before extracting the cannabinoids from the plant tissue powder. Extraction is usually performed by microwave extraction, supercritical CO₂ extraction or solvent extraction such as ethanol, glycerin or water. In a preferred embodiment, cannabinoids are extracted using ethanol or supercritical CO₂ extraction.

Step c)—Optional Purification of the One or More Decarboxylated Cannabinoid(s)

In optional step c), cannabinoids may optionally be further purified, depending on its their intended use.

For non-pharmaceutical uses, plant extracts may usually be used without further purification. However, for pharmaceutical uses, substantially purified compounds (substantially pure form typically contains at least 90%, or at least 95% or at least 99% of the component) are preferred, and the method for producing one or more decarboxylated cannabinoid(s) according to the invention may then comprise one or more purification steps.

A skilled person will be able to design appropriate purification steps, based on common general knowledge and the desired final purity.

For instance, suitable purification steps may include preparatory chromatography using normal phase or reverse phase columns (in particular a C18 reverse phase column), allowing the separation of the extract in different fractions. Fractions may be further purified by similar methods or by evaporation and resuspension of the compounds in a specific solvent, in which impurities are insoluble.

In a preferred embodiment, cannabinoids extracted with supercritical CO₂ in step b) are first winterized: the extract is dissolved in ethanol, stored at −20° C. until solidification of the co-extracted oil and waxes, and filtered to remove the oil and waxes. The extract is then further purified in the same manner as an extract obtained from ethanol extraction on plant tissue powder, preferably by using C18 reverse phase columns.

Step d)—Optional Addition of an Acceptable Diluent, Excipient or Carrier

In optional step d), an acceptable diluent, excipient or carrier may be added to the (optionally purified) one or more decarboxylated cannabinoid(s).

Suitable diluents, excipients, or carriers include, but are not limited to oils (notably pure, as micelles, or as microdroplets), cyclodextrins, or solvents such as ethanol, glycerin, and water.

Use of One or More Bacterial and/or Yeast Strain(s) for Decarboxylating One or More Cannabinoid(s) in Plant Tissues of Cannabis sativa Cultivars Cultivated in a Temporary Liquid Immersion Culture System

The present invention also relates to the use of one or more bacterial and/or yeast strain(s) for decarboxylating one or more cannabinoid(s) in plant tissues of Cannabis sativa cultivars cultivated in a temporary liquid immersion culture system, wherein the one or more bacterial and/or yeast strain(s) are co-cultivated with the Cannabis sativa cultivars in the temporary liquid immersion culture system.

Any preferred embodiment disclosed above in the context of the method for producing plant tissues containing one or more decarboxylated cannabinoid(s) according to the invention also applies to the use according to the invention.

The following examples merely intend to illustrate the present invention.

### EXAMPLES

### Example 1: Effect of In Vitro Co-Culture in TIB of Cannabis sativa Cultivars with Bacterial or Yeast Strains on the Proportion of Decarboxylated Cannabinoids

In this example, the inventors found out that some TIB in which Cannabis sativa cultivars were cultivated were contaminated by bacterial or yeast strains, while other TIB were not contaminated. Some of the contaminated TIB were further subjected to abiotic stress (drought) and the ratio of decarboxylated cannabinoids measured in the various conditions.

### Materials and Methods

Cannabis sativa Cultivars

The following Cannabis sativa cultivars were used: Jamaican Pearl (also referred to as “JP”), California indica (also referred to as “CL”), Cannatonic (also referred to as “CN”), and Amazing Haze (also referred to as “AH”). These cultivars were selected to represent a range of chemotypes, sizes, photoperiod responses and genetic backgrounds.

### Culture Conditions

Sterile Cannabis sativa apical meristems and nodal segments were multiplied in sterile liquid culture medium (DKW medium for Jamaican Pearl, California Indica and Amazing Haze; MS medium for Cannatonic) supplemented with 0.5 mg/L BAP. RITA® TIB were cultivated at 25° C. for biomass production during 4 weeks under 16 h light/8 h night photoperiod, light intensity was between 100 and 300 μmol photons/m²/s and 3 min immersions every 6 h were used.

### Decarboxylation Process

In experiments with Jamaican Pearl, California indica and Amazing Haze cultivars, and two out of three experiments with Cannatonic cultivar (corresponding to results of FIGS. 1-5), the decarboxylation process was performed as follows:

After 4 weeks, half of plant material present in the contaminated TIB was harvested. Also, all the plant material of non-contaminated TIB (control) were harvested at the same time. The second half of contaminated TIB was further treated with an abiotic stress (cessation of immersion) for 3 additional weeks in the same temperature and light conditions. This second part was thus harvested after 7 weeks of culture.

### Analysis of Cannabinoid Content Using HPLC

Extraction of cannabinoids: After freeze drying the dry biomass is ground with a vibrational shaker. 30 mg of dry material is extracted three times with 1 ml Ethanol 96%, filtered on 0.2 μm nylon filters and analyzed by HPLC.

HPLC analysis: Chromatographic separation is conducted on a Kinetex® Core Shell C18 column (Phenomenex, 2.6 μm, 100A, 150×3 mm) protected by a precolumn (Phenomenex, SecurityGuard™ ULTRA Cartridges, UHPLC C18 3.0 mm). The mobile phase is composed of A: water+0.1% Formic Acid (AF), B: Methanol+0.1% AF. Flow is set to 0.7 ml/min, and oven temperature at 50° C. 10 μl sample are injected. The separation gradient starts from 70% B, up to 85% B in 20 min, followed by 95% B for 5 min and back to 70% for 5 min. Detection is done with a Diode Array Detector at 230 nm. Quantification of main cannabinoids is done using 11 analytical standards (Merck-Sigma Aldrich): CBDV, THCV, CBD, CBG, CBDA, CBGA, CBN, THC, CBC, THCA, and CBCA.

Ratio/percentage of decarboxylated cannabinoids: the percentage of decarboxylated cannabinoids was calculated according to the following equation:

% of decarboxylated cannabinoid=(% of the cannabinoid in its neutral form)/(sum of % of cannabinoid in its acid and neutral forms)×100

### Microorganisms' Species Identification

Microorganisms were isolated from contaminated liquid cultures or contaminated plants. 1 ml of microorganism's suspension or 20 mg of contaminated plants are transferred to solid microorganism's culture medium (LB or TSA) and solid cannabis culture medium (MS or DKW). Plates are incubated at 25° C. or 37° C. until colonies become visible. To isolate a single colony, an inoculum of microorganism is streaked across an agar plate with an inoculating sterile loop. Sterilized loop is then passed once through the first streak and streaked across a fresh part of the plate. This process is repeated at least once more, and the plate is incubated at 25° C. or 37° C. until colonies become visible. The isolated microorganisms' colonies were used to identify the microorganism's species using matrix assisted laser desorption ionization-time of flight mass spectrometry (MALDI-TOF MS).

### Results

### Identified Microorganisms

Five different microorganisms were identified in five tests. Four different Cannabis sativa cultivars were analyzed. Of the six microorganisms, five bacteria and one yeast species were identified (see Table 2 below).

TABLE 2
Identified microorganisms detected in six different tests.

| Corresponding | Cannabis sativa | Identified |
|---|---|---|
| FIG. | cultivar | microorganisms |
| 1 | JP | Curtobacterium spp. |
| | | (Bacteria) |
| 2 | CL | Staphylococcus |
| | | epidermidis (Bacteria) |
| 3 | CN-B | Micrococcus luteus |
| | | (Bacteria) |
| 4 | CN-B | Microbacterium |
| | | ginsengisoli (Bacteria) |
| 5 | AH | Cystobasidium minutum |
| | | (Yeast) |

JP: Jamaican Pearl; CL: California indica; CN-B: Cannatonic 2; AH: Amazing Haze.

### Decarboxylation of Cannabinoids

Results obtained for the 5 conditions disclosed in Table 2 above are presented in FIGS. 1 to 5, and discussed in more details below for each condition.

### Condition 1: Cultivar Jamaican Pearl (JP)/Curtobacterium Spp. (Bacteria) (FIG. 1):

Two main types of cannabinoids were detected in this cultivar when the TIB was contaminated with Curtobacterium spp.: THCA and CBCA. 46.24% of THCA and 42.19% of CBCA were decarboxylated in the contaminated TIB further submitted to abiotic stress. Approximately 4% of neutral THC and 1% of CBC were already present in the control plant material produced in TIB without contamination and approximately 6% of neutral THC and 2% of CBC in TIB contaminated but not further submitted to abiotic stress.

Condition 2: Cultivar California Indica (CL)/Staphylococcus epidermidis (Bacteria)(FIG. 2):

Two main types of cannabinoids were detected in this cultivar when the TIB was contaminated with one or more Staphylococcus epidermidis: THCA and CBCA. 67.74% of THCA and 55.85% of CBCA were decarboxylated in the contaminated TIB further submitted to abiotic stress. Approximately 5% of neutral THC and 2% of CBC were already present in the control plant material produced in TIB without contamination and in TIB contaminated but not further submitted to abiotic stress.

Condition 3: Cultivar Cannatonic (CN-B)/Micrococcus luteus (Bacteria) (Graph 3):

Three main types of cannabinoids were detected in this cultivar when the TIB was contaminated with Micrococcus luteus: THCA, CBCA and CBDA. 89.95% of THCA, 60.72% of CBCA and 59.09% of CBDA were decarboxylated in the contaminated TIB further submitted to abiotic stress. Approximately 2% of neutral THC and 4% of CBD were already present in the control plant material produced in TIB without contamination and in TIB contaminated but not further submitted to abiotic stress. No CBC was detected in the control plant material.

Condition 4: Cultivar Cannatonic (CN-B)/Microbacterium ginsengisoli (Bacteria) (FIG. 4):

Three main types of cannabinoids were detected in this cultivar when the TIB was contaminated with Microbacterium ginsengisoli: THCA, CBCA and CBDA. 66.94% of THCA, 26.57% of CBCA and 36.80% of CBDA were decarboxylated in the contaminated TIB further submitted to abiotic stress. Approximately 3% of neutral THC and 5% of CBD were already present in the control plant material produced in TIB without contamination and in TIB contaminated but not further submitted to abiotic stress. No CBC was detected in the control plant material.

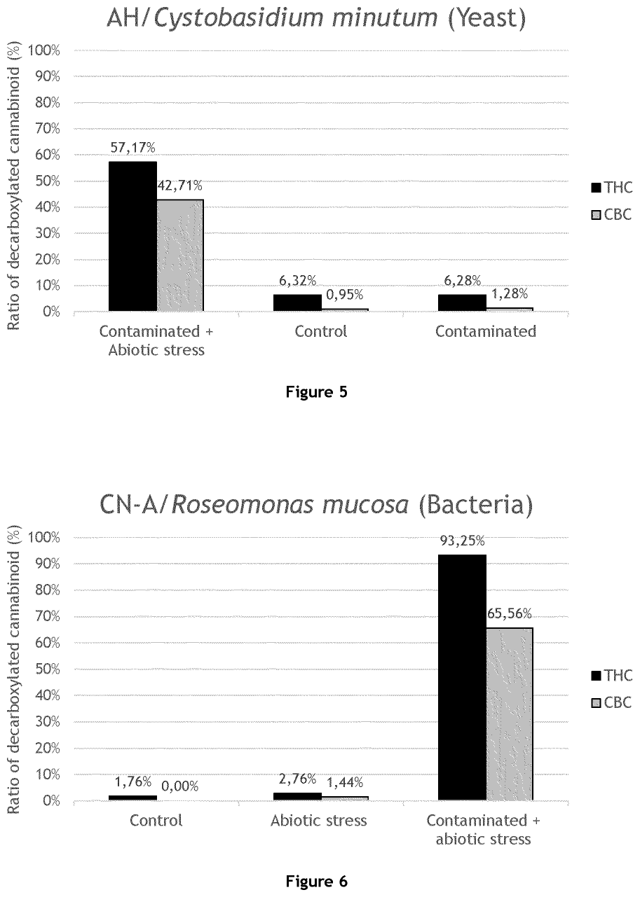

Condition 5: Cultivar Amazing Haze (AH)/Cystobasidium minutum (Yeast) (FIG. 5):

Two main types of cannabinoids were detected in this cultivar when the TIB was contaminated with Cystobasidium minutum: THCA and CBCA. 57.17% of THCA and 42.71% of CBCA were decarboxylated in the contaminated TIB further submitted to abiotic stress. Approximately 6% of neutral THC and 1% of CBC were already present in the control plant material produced in TIB without contamination and in TIB contaminated but not further submitted to abiotic stress.

### Conclusions

The above results suggest that the presence of bacteria or yeast in the culture medium of Cannabis sativa cultivars cultured in TIB for 7 weeks, of which 3 weeks were performed under abiotic stress, permits to obtain significant percentages of decarboxylated cannabinoids (THC, CBC and optionally CBD, depending on the cultivar used) in plant materials, without the need for further steps of decarboxylation using conventional methods (such as heating or microwaves).

Results are presented above for Jamaican Pearl, California indica, Cannatonic, and Amazing Haze cultivars and for Curtobacterium spp. (Bacteria), Staphylococcus epidermidis (Bacteria), Micrococcus luteus (Bacteria), Microbacterium ginsengisoli (Bacteria), and Cystobasidium minutum (Yeast), but decarboxylation of cannabinoids in plant tissues has also been observed for three additional cultivars (Afghani, Sensi 741 and Michka) cultivated in TIB contaminated by not yet identified microorganisms, or for additional microorganisms, including Roseomonas mucosa (see Example 2), Bacillus megaterium and Sphingomonas paucimobilis (data not shown).

### Example 2: Culture Under Abiotic Stress Improves Decarboxylation but is not Necessary to Decarboxylation

In this example, the inventors show that abiotic stress (drought) enhances decarboxylation activity in Cannabis sativa cultivars contaminated at different stages of cultivation. However, the results show that the application of abiotic stress is not necessary to trigger the decarboxylation process and is not able to induce a significant decarboxylation activity alone without the presence of microorganisms.

### Materials and Methods

Cannabis sativa Cultivar and Culture Conditions

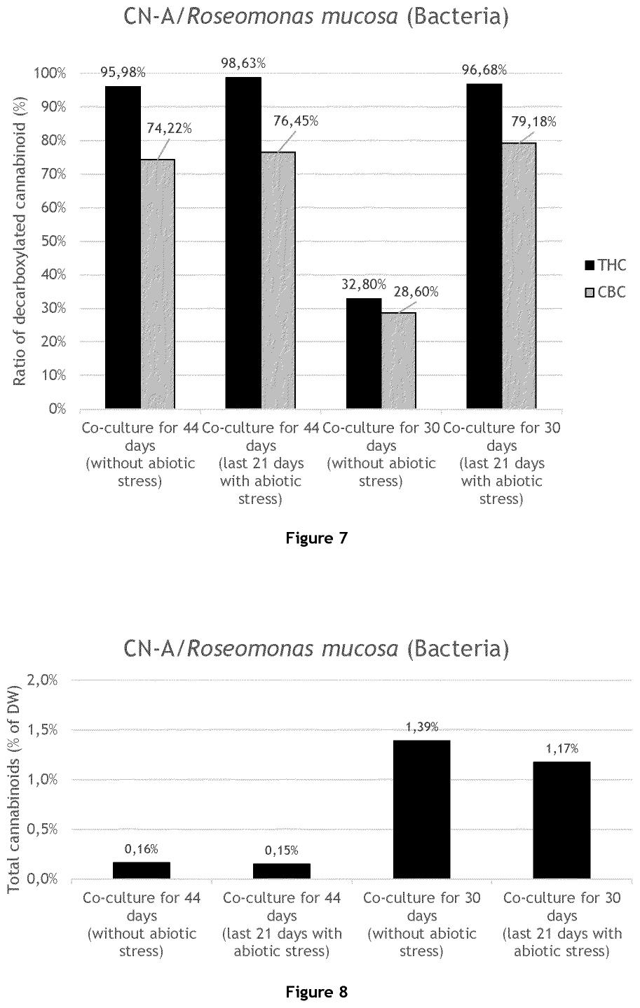

The following Cannabis sativa cultivar was used: Cannatonic (also referred to as “CN-A”). Sterile Cannabis sativa apical meristems and nodal segments were multiplied in sterile liquid culture MS (experiment shown in FIG. 6) or DKW (experiment shown in FIGS. 7 and 8) medium supplemented with 0.5 mg/L BAP. RITA® TIB were cultivated at 25° C. for biomass production during 4 weeks under 16 h light/8 h night photoperiod, light intensity was between 100 and 300 μmol photons/m²/s and 3 min immersions every 6 h were used.

### Decarboxylation Process

In the first experiment (corresponding to results of FIG. 6), the inventors found that one TIB was contaminated (and the contaminant identified for further experimentation). After 4 weeks, the plant material of a non-contaminated TIB (control) was harvested. All the plant material of other non-contaminated TIB and contaminated TIB is treated with an abiotic stress (cessation of immersion) for 3 additional weeks in the same temperature and light conditions and they were harvested after 7 weeks of culture.

In the second experiment (corresponding to results of FIGS. 7-8) the bacteria previously identified was applied for different periods of co-culture with the plants to trigger the decarboxylation process as follows:

At 5- or 19-days post inoculation of the TIB with sterile plant material, the liquid culture medium was inoculated with 880 μL or 500 μL of a bacterial solution at an optical density (OD) between 0.135 and 0.238. The final OD of the bacteria in the TIB medium was 0.0057. After 4 weeks, half of plant material present in the contaminated TIB was further treated with an abiotic stress (cessation of immersion) for 3 additional weeks in the same temperature and light conditions. The second part was harvested after 7 weeks of culture (44 or 30 days in presence of the bacteria). Also, all the plant material of non-contaminated TIB (control) were harvested at the same time.

### Analysis of Cannabinoid Content Using HPLC

See Example 1.

### Microorganisms' Species Identification

See Example 1.

### Results

### Identified Microorganisms

One microorganism was identified from the contaminated TIB presented in FIG. 6 and was used for the experiment of FIGS. 7 and 8: Roseomonas mucosa (Bacteria). (see Table 3 below).

TABLE 3
Identified microorganism used in two different tests.

| | | | Concentration of |
|---|---|---|---|
| | Cannabis | | microorganism in |
| Corresponding | sativa | Identified | contaminated |
| FIG. | cultivar | microorganisms | culture medium |
| 6 | CN-A | Roseomonas | undetermined |
| | | mucosa (Bacteria) | |
| 7-8 | CN-A | Roseomonas | 0.0057 (Final OD) |
| | | mucosa (Bacteria) | |

CN-A: Cannatonic.

### Decarboxylation of Cannabinoids

Results obtained for the 2 conditions disclosed in Table 3 above are presented in FIGS. 6 to 8, and discussed in more details below for each condition.

Condition 1: Cultivar Cannatonic (CN-A)/Roseomonas mucosa (Bacteria) (FIG. 6):

Two main types of cannabinoids were detected in this cultivar when the TIB was contaminated with Roseomonas mucosa: THCA and CBCA. 93.25% of THCA and 65.56% of CBCA were decarboxylated in the contaminated TIB subjected to abiotic stress. Approximately 2% of neutral THC and 1% of CBC were decarboxylated in the control plant material produced in TIB without contamination harvested after 4 weeks of plant culture or in TIB submitted to an additional 3 weeks abiotic stress (FIG. 6). No decarboxylation is thus observed after abiotic stress without contamination.

Condition 2: Cultivar Cannatonic (CN-A)/Roseomonas mucosa (Bacteria) (FIGS. 7 and 8):

Two main types of cannabinoids (THCA and CBCA) were detected in this cultivar when the TIB was inoculated with Roseomonas mucosa at two different times of the plant culture: 5 and 19 days after inoculation of plant material.

With the first contamination time, 98% of THCA and 74.22% of CBCA were decarboxylated after 44 days in regular co-culture conditions and 98.63 of THCA and 76.45 of CBCA were decarboxylated in the TIB submitted to 23 days in regular conditions followed by 21 days under abiotic stress.

When the TIB was contaminated after 19 days of plant culture, 32.80% of THCA and 28.60% of CBCA were decarboxylated with 30 days of co-culture and 96.68 of THCA and 79.18 of CBCA were decarboxylated in the TIB submitted to 9 days in regular conditions followed by 21 days under abiotic stress.

The rate of decarboxylation of THCA and CBCA is thus similar when the bacterial suspension is added at 5 days with and without abiotic stress and at 19 days only when plants are submitted to 3 weeks abiotic stress after 4 weeks of cultivation. Without this stress, the rate of decarboxylation is approximately 65% lower (FIG. 7).

The total content in cannabinoids is approximately of 0.16% of the dry weight biomass collected in the TIB contaminated at 5 days of cannabis culture submitted to abiotic stress or not. This percentage is higher when the bacteria are supplemented at 19 days of cannabis culture. The total cannabinoids content in the TIB contaminated at 19 days of cannabis culture is of 1.39% of dry weight biomass in regular growth conditions for 30 days and 1.17% after 9 days regular conditions submitted to 21 days abiotic stress. Bacteria supplemented at the beginning of cannabis in-vitro cultivation (5 days) have a negative impact on plant growth (data not shown) and total cannabinoid production compared to bacterial contamination at 19 days (FIG. 8).

### Conclusions

The results presented in the example 2 suggest that abiotic stress (drought) can enhance decarboxylation activity in Cannabis sativa. However, the results show that the presence of abiotic stress is not necessary to trigger the decarboxylation process and that alone, without the presence of microorganisms, it doesn't induce a significant decarboxylation of cannabinoids. The concentration and timing of the addition of the bacterial suspension must be adjusted to avoid loss of yield and cannabinoid production.

### BIBLIOGRAPHIC REFERENCES

Alvard D., Cote, F., and Teisson, C. (1993). Comparison of methods of liquid medium culture for banana micropropagation: effects of temporary immersion of explants. Plant Cell, Tissue and Organ Culture, 32, 55-60;
Berman P., Futoran K., Lewitus G. M., Mukha D., Benami M., Shlomi T., Meiri D. (2018). A new ESI-LC/MS approach for comprehensive metabolic profiling of phytocannabinoids in Cannabis. Sci Rep. 8(1), 14280.
Etienne, H., and Berthouly, M. (2002). Temporary immersion systems in plant micropropagation. Plant Cell Tissue Organ Cult. 69, 215-231;
Hazekamp, A., Simons, R., Peltenburg-Looman, A., Sengers, M., van Zweden, R., &t Verpoorte, R. (2004). Preparative isolation of cannabinoids from Cannabis sativa by centrifugal partition chromatography. Journal of liquid chromatography & related technologies, 27(15), 2421-2439;
Iffland, K., Carus, M., &t Grotenhermen, F. (2016). Decarboxylation of Tetrahydrocannabinolic acid (THCA) to active THC. European Industrial Hemp Association;
Lewis-Bakker, M. M., Yang, Y., Vyawahare, R., &t Kotra, L. P. (2019). Extractions of medical cannabis cultivars and the role of decarboxylation in optimal receptor responses. Cannabis and cannabinoid research, 4(3), 183-194;
Lewis-Bakker, M. M., Yang, Y., Vyawahare, R., &t Kotra, L. P. (2019). Extractions of medical cannabis cultivars and the role of decarboxylation in optimal receptor responses. Cannabis and cannabinoid research, 4(3), 183-194;
McPartland, J. M., MacDonald, C., Young, M., Grant, P. S., Furkert, D. P., &t Glass, M. (2017). Affinity and efficacy studies of tetrahydrocannabinolic acid A at cannabinoid receptor types one and two. Cannabis and cannabinoid research, 2(1), 87-95;
Pertwee, R. (2008). The diverse CB1 and CB2 receptor pharmacology of three plant cannabinoids: Δ9-tetrahydrocannabinol, cannabidiol and A9-tetrahydrocannabivarin. British journal of pharmacology, 153(2), 199-215;
Potter, D. J. (2014). A review of the cultivation and processing of cannabis (Cannabis sativa L.) for production of prescription medicines in the UK. Drug Test. Anal. 6, 31-38;
US20120046352;
US20190038663;
US20210100864;
Veress, T., Szanto, J. I., &r Leisztner, L. (1990). Determination of cannabinoid acids by high-performance liquid chromatography of their neutral derivatives formed by thermal decarboxylation: I. Study of the decarboxylation process in open reactors. Journal of Chromatography A, 520, 339-347;
Wang, M., Wang, Y. H., Avula, B., Radwan, M. M., Wanas, A. S., van Antwerp, J., . . . &t Khan, I. A. (2016). Decarboxylation study of acidic cannabinoids: a novel approach using ultra-high-performance supercritical fluid chromatography/photodiode array-mass spectrometry. Cannabis and cannabinoid research, 1(1), 262-271;
Watt, M. P. (2012). The status of temporary immersion system (TIS) technology for plant micropropagation. Afr. J. Biotechnol. 11, 14025-14035;
Welander, J. et al. Evaluation of a new vessel system based on temporary immersion system for micropropagation, Scientia Horticulturae, Volume 179, 2014, Pages 227-232;
WO2012146872A1;
WO2016092098;
WO2019006466;
WO2019006470;
WO96/25484;

1. A method for producing plant tissues containing one or more decarboxylated cannabinoid(s), the method comprising:
a) inoculating a temporary liquid immersion culture system containing a sterile liquid culture medium containing at least one cytokinin and/or at least one auxin with sterile plant material from a Cannabis sativa cultivar,
b) cultivating the plant material in the temporary liquid immersion culture system under conditions suitable for growing plant biomass,
c) inoculating the sterile liquid culture medium with one or more bacterial and/or yeast strain(s),
d) cultivating the plant material in the temporary liquid immersion culture system inoculated by the one or more bacterial and/or yeast strain(s), and
e) collecting plant tissues containing one or more decarboxylated cannabinoid(s).

2. The method of claim 1, wherein said temporary liquid immersion culture system is a temporary immersion bioreactor (TIB).

3. The method according to claim 1, wherein the sterile liquid culture medium is selected from MS medium, DKW medium, B5 medium, WPM medium, and SH medium.

4. The method according to claim 1, wherein:
a) said cytokinin is a natural or artificial cytokinin belonging to the adenine-type or the phenylurea-type, and/or
b) said sterile liquid culture medium comprises from 0.01 to 10 mg/L of said cytokinin.

5. The method according to claim 1, wherein:
a) said auxin is selected from naturally occurring auxins, or synthetic auxin analogues, and/or
b) the concentration of auxin in the sterile liquid culture medium is from 0.01 to 10 mg/L.

6. The method according to claim 1, wherein the duration and frequency of immersion of the plant material in the liquid culture medium in step b) varies from 30 seconds to 15 minutes every 30 minutes to 12 hours.

7. The method according to claim 1, wherein in step b):
a) white light, red light, blue light or a mixture of red and blue lights is used;
b) a photoperiod of 12 to 24 hours of light and 0 to 12 hours of darkness is used; and/or
c) light intensity is between 50 and 600 pmol photons/m²/s of Photosynthetically Active Radiations (PAR.

8. The method according to claim 1, wherein the abiotic stress condition used in step d) is drought.

9. The method according to claim 1, wherein:
a) the one or more bacterial strain(s) is(are) selected from
i. The Actinomycetia, Alphaproteobacteria, and Bacilli classes;
ii. The Micrococcales, Rhodospirillales, Bacillales, and Sphingomonadales orders;
iii. The Microbacteriaceae, Micrococcaceae, Acetobacteraceae, Staphylococcaceae, Sphingomonadaceae, and Bacillaceae families;
iv. The Curtobacterium, Microbacterium, Micrococcus, Roseomonas, Staphylococcus, and Bacillus genus; or
v. The Curtobacterium genus, Microbacterium ginsengisoli, Micrococcus luteus, Roseomonas mucosa, Staphylococcus epidermis, Sphingomonas paucimobilis, and Bacillus megaterium species; or
b) the one or more yeast strain(s) is(are) selected from the Cystobasidiomycetes class, the Cystobasidiales order, the Cystobasidiaceae family, the Cystobasidium genus, or the Cystobasidium minutum species.

10. The method according to claim 1, wherein the optical density (DO) at 600 nm of the one or more bacterial and/or yeast strain(s) in the liquid culture medium after inoculation in step c) is between 0.0001 and 0.1.

11. The method according to claim 1, wherein the plant material comprises apical meristems and nodal segments from surface-sterilized seeds germinated in vitro.

12. The method according to claim 1, wherein the Cannabis sativa cultivar is selected from Jamaican Pearl cultivar, Cannatonic cultivar, California indica cultivar, Amazing Haze cultivar, Michka cultivar, Sensi 741 cultivar, and Afghani cultivar.

13. The method according to claim 1, wherein the one or more decarboxylated cannabinoid (s) is(are) selected from tetrahydrocannabinol (THC), cannabichromene (CBC), cannabidiol (CBD), and cannabigerol (CBG).

14. A method for producing one or more decarboxylated cannabinoid(s), the method comprising:
a) producing plant tissues containing one or more decarboxylated cannabinoid (s) using the method according to claim 1, and
b) collecting or extracting the one or more decarboxylated cannabinoid(s) from the obtained plant tissues.

15. A method for decarboxylating one or more cannabinoid(s) in plant tissues of Cannabis sativa cultivars cultivated in a temporary liquid immersion culture system, wherein the method comprises a step of a co-cultivating the one or more bacterial and/or yeast strain(s) with the Cannabis sativa cultivars in the temporary liquid immersion culture system.

16. The method according to claim 2, wherein the volume of sterile liquid culture medium in the TIB is from 1 L to 10,000 L.

17. The method according to claim 4, wherein said cytokinin is selected from adenine, kinetin, zeatin, 6-benzylaminopurine, diphenylurea, thidiazuron (TDZ), and derivatives thereof having cytokinin activity.

18. The method according to claim 5, wherein:
a1) the naturally occurring auxins are selected from 4-chloro-indoleacetic acid, phenylacetic acid (PAA), indole-3-butyric acid (IBA), and indole-3-acetic acid (IAA); or
a2) the synthetic auxin analogues are selected from 1-naphthaleneacetic acid (NAA) and 2,4-dichlorophenoxyacetic acid (2,4-D).

19. The method according to claim 8, wherein the abiotic stress condition used in step d) is drought caused by decreasing the duration and/or frequency of immersion of the plant material in the liquid culture medium or by stopping the immersion of the plant material in the liquid culture medium in step d).

20. The method for producing one or more decarboxylated cannabinoid(s) according to claim 14, further comprising the steps of:
c) purifying the collected or extracted one or more decarboxylated cannabinoid(s), and
d) adding an acceptable diluent, excipient or carrier to the one or more decarboxylated cannabinoid(s).
